(19) United States
(12) Patent Application Publication
(10) Pub. No.: US 2025/0154334 A1
(43) Pub. Date: May 15, 2025

(54) EFFICIENT CHEMICAL RECYCLING METHOD FOR WASTE POLYESTER FABRICS

(71) Applicant: NAN YA PLASTICS CORPORATION, Taipei City (TW)

(72) Inventors: Te-Chao Liao, Taipei City (TW); Jung-Jen Chuang, Taipei City (TW); Zhang-Jian Huang, Taipei City (TW); Cheng Hsun Chen, Taipei City (TW); Mong-Chen Chang, Taipei City (TW)

(73) Assignee: NAN YA PLASTICS CORPORATION, Taipei City (TW)

(21) Appl. No.: 18/530,162

(22) Filed: Dec. 5, 2023

(30) Foreign Application Priority Data
Nov. 10, 2023 (TW) 112143356

(51) Int. Cl. C08J 11/24

(52) U.S. Cl. CPC C08J 11/24; C08J 2367/02

(57) ABSTRACT

The disclosure provides a chemical recycling method for polyester fabrics, which includes the following steps. The polyester fabric is extracted using a composite solvent and filtered to obtain a decolored polyester fabric. The composite solvent contains alcohol ether and phenyl ether, and the decolored polyester fabric contains the composite solvent. Ethylene glycol is then added to the decolored polyester fabric to depolymerize into ethylene terephthalate monomers.

### CROSS-REFERENCE TO RELATED APPLICATION

This application claims the priority benefit of Taiwan application serial no. 112143356, filed on Nov. 10, 2023. The entirety of the above-mentioned patent application is hereby incorporated by reference herein and made a part of this specification.

### BACKGROUND

### Technical Field

The disclosure relates to an efficient chemical recycling method for fabrics, and particularly relates to an efficient chemical recycling method for waste polyester fabrics.

### Description of Related Art

In the conventional polyester fabric recycling method, the pre-treatment process usually first decolorizes the waste PET fabric to achieve the decolorization effect, including using a solvent to extract the dye, and then drying the decolored PET fabric to remove the solvent. The dried PET fabric is then chemically recycled. In the pre-treatment process, the fabric drying process requires heating to evaporate the solvent, and the evaporated solvent is condensed and recovered. This process has disadvantages, including (1) the solvent remaining on the fabric must be less than 1.0% to avoid affecting PET depolymerization, (2) drying requires equipment and energy consumption and (3) the solvent recovery process will cause solvent leakage.

After decolorization, the PET fabric is depolymerized with EG (ethylene glycol) to obtain the BHET monomer (Bis(2-HydroxyEthyl) Terephthalate) product, and purified to obtain the clean BHET monomer, which is generally called “the BHET monomer chemical recovery process”. In the conventional BHET monomer chemical recovery process, depolymerization is carried out at 190° C. to 240° C. The disadvantages of this process include (1) high temperature, (2) long depolymerization time, (3) low selectivity, and (4)) yellowish hue of BHET.

Based on the above, the conventional pre-treatment process and BHET monomer chemical recovery process have problems such as high cost and poor quality. Therefore, developing an efficient chemical recycling method for waste polyester fabrics, which solves the problems of high cost and poor quality in the conventional technology, is an important topic currently required for research.

### SUMMARY

The disclosure provides a chemical recycling method for waste polyester fabrics, which effectively solves the problems of high cost and poor quality in the conventional technology.

A chemical recycling method for polyester fabrics according to the disclosure includes the following. The polyester fabric is extracted using a composite solvent and filtered to obtain a decolored polyester fabric. The composite solvent contains alcohol ether and phenyl ether, and the decolored polyester fabric contains the composite solvent. Ethylene glycol is then added to the decolored polyester fabric to depolymerize into ethylene terephthalate monomers.

In an embodiment of the disclosure, alcohol ether includes ethylene glycol methyl ether, ethylene glycol ethyl ether, ethylene glycol propyl ether, ethylene glycol butyl ether, ethylene glycol phenyl ether, propylene glycol methyl ether, propylene glycol ethyl ether, propylene glycol propyl ether, propylene glycol butyl ether, propylene glycol phenyl ether or combinations thereof, Phenyl ether includes anisole, phenethyl ether, phenylpropyl ether, butyl ether, methyl anisole, methyl phenethyl ether, methyl phenyl ether, methyl phenyl ether or a combination thereof.

In an embodiment of the disclosure, a weight ratio of the composite solvent to the polyester fabric is 3:10 to 10:10.

In an embodiment of the disclosure, the extraction is performed at a temperature of 110° C. to 150° C.

In an embodiment of the disclosure, an extraction time ranges from 10 minutes to 60 minutes.

In an embodiment of the disclosure, the extraction is performed 2 times to 6 times.

In an embodiment of the disclosure, in the composite solvent, a weight ratio of alcohol ether to phenyl ether is 1:9 to 9:1.

In an embodiment of the disclosure, a weight ratio of ethylene glycol to the decolored polyester fabric is 2:1 to 6:1.

In an embodiment of the disclosure, catalysts for depolymerization include organic metals and ionic liquids, the organic metals include zinc acetate, organic titanium, organic antimony or organic aluminum.

In an embodiment of the disclosure, a weight ratio of the catalysts for depolymerization to the decolored polyester fabric is 0.5:100 to 10:100.

In an embodiment of the disclosure, a temperature of depolymerization is 140° C. to 190° C.

In an embodiment of the disclosure, a time of depolymerization is 1.5 hours to 5 hours.

In an embodiment of the disclosure, the ionic liquids include 1-butyl-3-methylimidazolium hexa-fluoro-phosphate (BMI-PF6) and 1-butyl-3-methylimidazolium tetra-fluoro-borate (BMI-BF4).

Based on the above, the disclosure provides a chemical recycling method for polyester fabrics, using a composite solvent (alcohol ether and phenyl ether) as a solvent for pre-treatment of waste PET fabrics, and using the composite solvent as a co-solvent for depolymerization of BHET monomer chemical recycling process. The composite solvent has the functions of a solvent for pre-treatment extraction and a co-solvent for depolymerization. Therefore, it can effectively solve the problems of high cost and poor quality in the conventional technology.

### DESCRIPTION OF THE EMBODIMENTS

Hereinafter, embodiments of the disclosure will be described in detail. However, these embodiments are illustrative, and the disclosure is not limited thereto.

In the present specification, a range represented by “a numerical value to another numerical value” is a schematic representation for avoiding listing all of the numerical values in the range in the specification. Therefore, the recitation of a specific numerical range covers any numerical value in the numerical range and a smaller numerical range defined by any numerical value in the numerical range, as is the case with any numerical value and a smaller numerical range thereof in the specification.

A chemical recycling method for polyester fabrics according to the disclosure includes the following. The polyester fabric is extracted using a composite solvent and filtered to obtain a decolored polyester fabric. The composite solvent contains alcohol ether and phenyl ether, and the decolored polyester fabric contains the composite solvent. Ethylene glycol is then added to the decolored polyester fabric to depolymerize into ethylene terephthalate monomers.

In the present embodiment, a complex solvent is used for the extraction of polyester fabrics. Polyester fabrics are, for example, waste polyester fabrics, which may include waste clothing, scraps from textile factories or unqualified products. The PET content of waste polyester fabrics is, for example, more than 90 wt %, and impurities such as dyes and glue are less than 10 wt %. The composite solvent contains alcohol ether and phenyl ether. The alcohol ether can include ethylene glycol methyl ether, ethylene glycol ethyl ether, ethylene glycol propyl ether, ethylene glycol butyl ether, ethylene glycol phenyl ether, propylene glycol methyl ether, propylene glycol ethyl ether, and propylene glycol. propyl ether, propylene glycol butyl ether, propylene glycol phenyl ether or combinations thereof. Phenyl ether may include anisole, phenylethyl ether, phenylpropyl ether, phenylbutyl ether, methyl anisole, methyl phenyl ether, methyl phenyl ether, methyl butyl ether or a combination thereof, but the disclosure is not limited thereto. More specifically, a weight ratio of alcohol ether to phenyl ether is, for example, 1:9 to 9:1, preferably 2:8 to 8:2; a weight ratio of composite solvent to polyester fabric is, for example, 3:10 to 10:10, preferably 4:10 to 9:10. In terms of the operating conditions of extraction, a temperature for extraction is, for example, 110° C. to 150° C., preferably 120° C. to 140° C.; a time for extraction is, for example, 10 minutes to 60 minutes, preferably 20 minutes to 40 minutes. The number of times of extraction is, for example, 2 to 6 times, preferably 3 to 5 times.

In the present embodiment, the polyester fabric is extracted using a composite solvent and filtered to obtain a decolored polyester fabric. The decolored polyester fabric contains the composite solvent. After decolorization, the fabric does not need to be dried and continues to be depolymerized with ethylene glycol into crude ethylene terephthalate monomers. In more detail, a weight ratio of ethylene glycol to the decolored polyester fabric is, for example, 2:1 to 6:1, and preferably, it is 3:1 to 4:1. Depolymerization catalysts may include organic metals and ionic liquids. The organic metals may include zinc acetate, organic titanium, organic antimony or organic aluminum. The ionic liquid may include 1-butyl-3-methylimidazole hexafluorophosphate (1-butyl-3-methylimidazolium hexa-fluoro-phosphate (BMI-PF6) and 1-butyl-3-methylimidazolium tetra-fluoro-borate (BMI-BF4). In terms of depolymerization operating conditions, a weight ratio of the depolymerization catalyst to the decolored polyester fabric is, for example, 0.5:100 to 10:100, preferably 1:100 to 5:100; the depolymerization temperature is, for example, 140° C. to 190° C., preferably 150° C. to 180° C.; the depolymerization time is, for example, 1.5 hours to 5 hours, preferably 2 hours to 4 hours.

In the present embodiment, a selectivity of ethylene terephthalate monomer is, for example, 90% or more. The crude ethylene terephthalate monomer product undergoes purification procedures such as activated carbon adsorption of impurities, crystallization, filtration and drying. The purified ethylene terephthalate monomer is then polymerized into r-PET. The quality of r-PET is L>62%, a±1.0, b±2.0.

The chemical recycling method for polyester fabrics according to the disclosure is described below in detail by way of experimental examples. However, the following experimental examples are not intended to limit the disclosure.

### Experimental Example

In order to verify that the chemical recycling method for polyester fabrics according to the disclosure achieves both separation and decolorization, and effectively solves the problems of high cost and poor quality in the conventional technology, the experimental examples are provided as follows.

### Example 1

103 g waste PET fabric (L=22.5%, a=4.4, b=5.6) was taken, of which impurities such as dye account for 3 g and PET material accounts for 100 g.

### (1) Pre-Processing Process:

Place the waste PET fabric into a 1 L three-neck glass flask, and pour 600 g of composite solvent A (ethylene glycol ethyl ether/ethylene glycol butyl ether/propylene glycol methyl ether/anisole/methylphenylene ether=2/1/3/2/2, weight ratio), heat to 128° C. and maintain the temperature for 0.5 hr, and then filter with an air extraction bottle to separate the fabric from the composite solvent A. This was the first extraction procedure. The filtered wet-based fabric (175 g) contained 101 g of fabric (including dyes, etc.) and 74 g of composite solvent.

Then put the wet-based fabric back into the three-neck glass flask, add 526 g of the composite solvent, heat it to 128° C. and maintain the temperature for 0.5 hr, and then filter it to separate the fabric from the composite solvent, which was the second extraction procedure. The third extraction procedure was the same as the second one. After three extractions of waste PET fabric, L=85.4%, a=1.7, b=2.3, wet-based decolorized fabric (174 g, composite solvent/fabric=74/100). The composite solvent A (alcohol ether+phenyl ether) was a co-solvent for PET depolymerization of BHET monomer chemical recovery process. Therefore, it did not require drying before entering the PET depolymerization process.

### (2) BHET Monomer Chemical Recovery Process:

Put the wet-baseD fabric into a three-neck glass flask, then add 26 g of composite solvent, 300 g of ethylene glycol (EG), and 1 g of zinc acetate as a catalyst. After heating to 150° C. and maintaining for 2.5 hours, PET was depolymerized into a BHET crude product, in which the conversion rate of PET=100%, the selectivity of BHET monomer (m-BHET) was 90.4%, and the selection rate of by-products such as BHET oligomers (dimers or trimers or more, o-BHET) was 10.6%.

Lower the temperature of the crude product from 150° C. to 18° C. to crystallize the BHET monomer, and then filter it with a 5 μm filter to obtain a wet-based BHET monomer filter cake, and then put the wet-based BHET filter cake into a 1 L three-neck glass flask, add 518 g of pure water, stir and heat to 90° C. for 0.5 hr, then filter with a 1 μm filter to remove BHET oligomers. Add 1 g of powdered activated carbon to the filtrate at 90° C. to absorb impurities and filter with a 0.5 μm filter, and the filtrate was cooled from 90° C. to 5° C. to crystallize the BHET monomer, then filtered with a 1 μm filter to obtain the BHET monomer crystals, and then dried with hot air at 70° C. The BHET moisture content was lower than 1% to facilitate repolymerization.

### (3) Repolymerization Process:

BHET monomer undergoes polycondensation reaction to obtain r-PET, with hue quality L=66.4%, =1.1, b=1.7.

A composite solvent is used as the extraction solvent for pre-treatment and the co-solvent for depolymerization, the fabric pre-treatment process does not require drying, and the production line does not need to invest in drying equipment, which saves energy consumption of 30 to 150 cal/g of fabric; in the depolymerization unit, the depolymerization temperature can be lowered and the selectivity is high, and the production line has the advantages of low energy consumption and high productivity; in terms of quality, the recycled r-PET has the advantages of high selectivity and low hue quality.

### Example 2 to Example 6

Respectively change the composite solvent formula, dosage, depolymerization catalyst type and amount, depolymerization added EG amount and depolymerization temperatures after PET fabric pretreatment (please refer to Table 1). The rest is the same as Example 1. The recovery rate of r-PET and hue quality are shown in Table 1.

The compound solvent formula is as follows:

Example 2 used composite solvent B: (ethylene glycol ethyl ether/ethylene glycol phenyl ether/propylene glycol methyl ether/anisole/methyl phenyl ether=1/2/3/2/2, weight ratio).

Example 3 used composite solvent C: (ethylene glycol butyl ether/propylene glycol methyl ether/propylene glycol phenyl ether/phenylene ether/methyl anisole=3/2/1/2/2, weight ratio).

Example 4 used composite solvent D: (ethylene glycol ethyl ether/propylene glycol ethyl ether/phenylene ether/methylphenylene ether=3/3/2/2, weight ratio).

Example 5 used composite solvent E: (ethylene glycol ethyl ether/ethylene glycol propyl ether/propylene glycol propyl ether/phenyl butyl ether/methyl phenyl propyl ether=2/1/3/2/2, weight ratio).

Example 6 used composite solvent F: (ethylene glycol methyl ether/ethylene glycol ethyl ether/propylene glycol butyl ether/phenyl propyl ether/methyl phenyl butyl ether=2/1/3/2/2, weight ratio)

As shown in Table 1, Example 1 to Example 6 which used the chemical recycling method of polyester fabric of the disclosure had a high selectivity of BHET monomer (more than 90.0%), and the L/a/b of the recovered r-PET=above 62. %/±1.0/±2.0, with the advantage of good color. In addition, the fabric pre-treatment process does not require drying, the production line does not need to invest in drying equipment, and no drying energy is consumed, which has the advantages of energy saving and cost reduction.

TABLE 1

| Example | | | Example 1 | Example 2 | Example 3 | Example 4 | Example 5 | Example 6 |
|---|---|---|---|---|---|---|---|---|
| Pre- | Extraction solvent | | Composite | Composite | Composite | Composite | Composite | Composite |
| processing | | | solvent A | solvent B | solvent C | solvent D | solvent E | solvent F |
| process | Extraction | | 600/103 | 600/103 | 600/103 | 600/103 | 400/103 | 1000/103 |
| | solvent/fabric | | | | | | | |
| | (w/w) | | | | | | | |
| | Extraction | | 128 | 110 | 140 | 128 | 140 | 150 |
| | temperature (° C.) | | | | | | | |
| | Extraction time | | 30 | 30 | 20 | 60 | 40 | 30 |
| | (minutes) | | | | | | | |
| | Extraction times | | 3 | 6 | 3 | 4 | 5 | 2 |
| | Hue | L(%) | 88.3 | 87.4 | 90.1 | 89.0 | 88.1 | 91.3 |
| | | a | 0.5 | 0.7 | 0.3 | 0.4 | 0.4 | 0.3 |
| | | b | 0.9 | 1.1 | 0.7 | 0.8 | 0.7 | 0.7 |
| | Fabric drying | | No | No | No | No | No | No |
| | Extraction | | 74/100 | 30/100 | 54/98 | 100/99 | 76/99 | 45/92 |
| | solvent/fabric | | | | | | | |
| | (g/g) | | | | | | | |
| | Fabric drying | | 0.0 | 0.0 | 0.0 | 0.0 | 0.0 | 0.0 |
| | energy | | | | | | | |
| | consumption | | | | | | | |
| | (cal/g) | | | | | | | |
| | Extraction solvent | | 0.0 | 0.0 | 0.0 | 0.0 | 0.0 | 0.0 |
| | lost during fabric | | | | | | | |
| | drying (g/g fabric) | | | | | | | |
| BHET | Fabric after | | 100 | 100 | 98 | 99 | 99 | 92 |
| monomer | pretreatment (g) | | | | | | | |
| chemical | Fabric after | | 74 (composite | 30 (composite | 54 (composite | 100 (composite | 76 (composite | 45 (composite |
| recovery | pretreatment | | solvent)/100 | solvent)/100 | solvent)/98 | solvent)/99 | solvent)/99 | solvent)/96 |
| process | {Composite | | | | | | | |
| | solvent/fabric | | | | | | | |
| | (g/g)} | | | | | | | |
| | Supplement | | 26 | 70 | 146 | 100 | 74 | 55 |
| | extraction solvent | | | | | | | |
| | (g) | | | | | | | |
| | Ethylene glycol | | 300 | 300 | 600 | 200 | 400 | 300 |
| | (g) | | | | | | | |
| | Zinc acetate | | 1.0 | 2.0 | 2.0 | 3.0 | 5.0 | 4.0 |
| | catalyst (g) | | | | | | | |
| | Depolymerization | | 150 | 160 | 140 | 190 | 170 | 180 |
| | temperature (° C.) | | | | | | | |
| | Depolymerization | | 2.5 | 2.5 | 3.0 | 3.0 | 3.0 | 3.0 |
| | time (hr) | | | | | | | |
| | BHET monomer | | 93.4 | 92.7 | 92.1 | 90.1 | 91.3 | 90.8 |
| | selectivity of | | | | | | | |
| | depolymerized | | | | | | | |
| | crude product (%) | | | | | | | |
| Repolymerization | r-PET | L(%) | 66.2 | 65.8 | 67.3 | 62.1 | 65.3 | 63.8 |
| process | hue | a | 0.1 | 0.6 | 0.3 | 0.7 | 0.6 | 0.4 |
| | | b | 0.8 | 1.1 | 1.3 | 1.9 | 1.7 | 1.4 |

### Comparative Example 1

Take 103 g of waste PET fabric (L=22.5%, a=4.4, b=5.6), of which impurities such as dye account for 3 g and PET material accounts for 100 g.

### (1) Pre-Processing Process:

Place the waste PET fabric into a 1 L three-neck glass flask, pour 600 g of o-xylene, heat to 128° C. and maintain the temperature for 0.5 hr, then filter with an air extraction bottle to separate the fabric from the o-xylene. For the first extraction procedure, the filtered wet fabric (164 g) contained 101 g of fabric (including dyes, etc.) and 63 g of o-xylene.

Put the wet fabric back into the three-neck glass flask, add 537 g of o-xylene, heat to 128° C. and keep the temperature for 0.5 hr, and then filter to separate the fabric from o-xylene, which was the second extraction. The third extraction procedure was the same as the second one. After three extractions of waste PET fabrics, L=86.1%, a=1.9, =2.1, the wet-based decolorized fabric (169 g, xylene/fabric=69/100) was dried with a dryer, and the o-xylenes of the fabric was reduced to 0.5 g/100 g fabric to facilitate the subsequent chemical recovery process of BHET monomer. In the drying process, the wet-based decolorized fabric was heated at 125° C. The o-xylene was vaporized by heat and separated from the fabric. The vaporized o-xylene was then condensed to recover 58.1 g of o-xylene. The o-xylene loss rate was 10.9 g./100 g fabric.

### (2) BHET Monomer Chemical Recovery Process:

Put the dried fabric into a three-neck glass flask, add 400 g of ethylene glycol (EG) and 1 g of zinc acetate as a catalyst, heat to 195° C. and maintain for 4 hours, depolymerize PET into crude BHET product, in which the conversion of PET rate=100%, the selectivity of BHET monomer (m-BHET) was 79.2%, and the selectivity of by-products such as BHET oligomers (dimers or trimers, o-BHET) was 20.8%.

Lower the temperature of the crude product from 195 to 18° C. to crystallize the BHET monomer, then filter it with a 5 μm filter to obtain a wet BHET monomer filter cake, and then put the wet BHET filter cake into a 1 L three-neck glass flask, add 518 g of pure water, stir and heat to 90° C. for 0.5 hr, then filter with a 1 μm filter to remove BHET oligomers, add 1 g of powdered activated carbon to the filtrate at 90° C. to absorb impurities and filter with 0.5 μm, and the filtrate is then cooled from 90° C. to 5° C. to crystallize the BHET monomer, then filter it with a 1 μm filter to obtain the crystals of the BHET monomer, and then dry it with hot air at 70° C. The moisture content of BHET was less than 1% to facilitate repolymerization.

### (3) Repolymerization Process:

BHET monomer undergoes polycondensation reaction to obtain r-PET, with hue quality L=60.4%, =1.9, b=3.7.

Using xylene as the extraction solvent for pretreatment and no co-solvent during depolymerization, the test results are shown in Table 2. The BHET monomer selectivity is low (79.2%), and the L/a/b of the recovered r-PET=60.4%/1.9/3.7, with shortcomings such as color difference. In addition, the fabric pre-treatment process requires drying, the production line needs to invest in drying equipment and use 55 cal/g of fabric, and the leakage rate of recycled o-xylene=0.019 g/g of fabric.

The yield and hue quality of r-PET in Comparative Examples 2 to 6 are shown in Table 2. The descriptions of Comparative Examples 2 to 6 are as follows.

### Comparative Example 2

The fabric was not dried in the pre-treatment process, and the rest was the same as Comparative Example 1. The BHET monomer selectivity is low (76.4%); the recycled r-PET has L/a/b=61.4%/2.4/4.2, which has shortcomings such as color difference.

### Comparative Example 3

The depolymerization temperature of the BHET monomer chemical recovery process was changed to 150° C., and the rest was the same as Comparative Example 1. BHET monomer has low selection (45.8%), and the recycled r-PET has L/a/b=60.8%/1.5/3.4, which has shortcomings such as color difference. In addition, the fabric pre-treatment process requires drying, the production line needs to invest in drying equipment and use 55 cal/g of fabric, and the leakage rate of recycled o-xylene=0.019 g/g of fabric.

### Comparative Example 4

The EG in the BHET monomer chemical recovery process was reduced from 400 g to 300 g, and 100 g anisole was added for depolymerization. The rest was the same as Comparative Example 3. Although the selectivity of BHET monomer is high (90.3%), the recycled r-PET has L/a/b=64.1%/1.0/1.7 and good color. However, the fabric pre-treatment process requires drying, and the production line requires investment in drying equipment and an energy consumption of 55 cal/g of fabric, and the leakage rate of recycled xylene=0.019 g/g of fabric.

### Comparative Example 5

Extraction solvent/fabric 1,000/103 g, extraction temperature 140° C., extraction time 60 minutes, extraction times 6 times, catalyst 2 g, depolymerization temperature 150° C., the rest was the same as Comparative Example 3. BHET monomer has low selection (86.7%), the recycled r-PET has L/a/b=63.7%/1.1/1.5, and the color is good.

### Comparative Example 6

Ethylene glycol was used instead of o-xylene as the extraction solvent for pretreatment, and 100 g of anisole was added during depolymerization. The rest was the same as Comparative Example 2. BHET monomer selectivity is high (91.4%); however, the recycled r-PET has L/a/b=55.4%/2.5/6.1, which has the disadvantage of hue difference.

TABLE 2

| | | | Comparative | Comparative | Comparative | Comparative | Comparative | Comparative |
|---|---|---|---|---|---|---|---|---|
| Comparative Example | | | Example 1 | Example 2 | Example 3 | Example 4 | Example 5 | Example 6 |
| Pre- | Extraction solvent | | O-xylene | O-xylene | O-xylene | O-xylene | O-xylene | Ethylene |
| processing | | | | | | | | glycol |
| process | Extraction | | 600/103 | 600/103 | 600/103 | 600/103 | 1,000/103 | 600/103 |
| | solvent/fabric | | | | | | | |
| | (w/w) | | | | | | | |
| | Extraction | | 128 | 128 | 128 | 128 | 140 | 128 |
| | temperature (° C.) | | | | | | | |
| | Extraction time | | 30 | 30 | 30 | 30 | 30 | 60 |
| | (minutes) | | | | | | | |
| | Extraction times | | 3 | 3 | 3 | 3 | 6 | 3 |
| | Hue | L(%) | 86.1 | 86.1 | 86.1 | 86.1 | 91.4 | 77.2 |
| | | a | 1.4 | 1.4 | 1.4 | 1.4 | 0.9 | 2.1 |
| | | b | 1.6 | 1.6 | 1.6 | 1.6 | 1.1 | 4.3 |
| | Fabric drying | | Yes | No | Yes | Yes | No | No |
| | Extraction | | 0.5/100 | 30/100 | 0.5/100 | 0.5/100 | 30/100 | 30/100 |
| | solvent/fabric | | | | | | | |
| | (g/g) | | | | | | | |
| | Fabric drying | | 55 | 0.0 | 55 | 55 | 55 | 0.0 |
| | energy | | | | | | | |
| | consumption | | | | | | | |
| | (cal/g) | | | | | | | |
| | Extraction solvent | | 10.9/100 | 0.0/100 | 10.9/100 | 10.9/100 | 0.0/100 | 0.0/100 |
| | lost during fabric | | | | | | | |
| | drying (g/g fabric) | | | | | | | |
| BHET | Fabric after | | 100 | 100 | 100 | 100 | 98 | 100 |
| monomer | pretreatment (g) | | | | | | | |
| chemical | Fabric after | | 0.5(xylene)/100 | 30(xylene/100 | 0.5(xylene)/100 | 0.5(xylene)/100 | 30(xylene/100 | 30(Ethylene |
| recovery | pretreatment | | | | | | | glycol)/100 |
| process | {Composite | | | | | | | |
| | solvent/fabric | | | | | | | |
| | (g/g)} | | | | | | | |
| | Supplement | | 0 | 0 | 0 | 100 | 100 | 100 |
| | extraction solvent | | | | | | | |
| | (g) | | | | | | | |
| | Ethylene glycol | | 400 | 400 | 400 | 300 | 400 | 370 |
| | (g) | | | | | | | |
| | Zinc acetate | | 1.0 | 1.0 | 1.0 | 1.0 | 2.0 | 3.0 |
| | catalyst (g) | | | | | | | |
| | Depolymerization | | 195 | 195 | 150 | 150 | 150 | 195 |
| | temperature (° C.) | | | | | | | |
| | Depolymerization | | 4.0 | 4.0 | 4.0 | 4.0 | 4.0 | 4.0 |
| | time (hr) | | | | | | | |
| | BHET monomer | | 79.2 | 76.4 | 45.8 | 90.3 | 86.7 | 91.4 |
| | selectivity of | | | | | | | |
| | depolymerized | | | | | | | |
| | crude product (%) | | | | | | | |
| Repolymerization | Hue | L(%) | 60.4 | 61.2 | 60.8 | 64.1 | 63.7 | 55.4 |
| process | | a | 1.9 | 2.4 | 1.5 | 1.0 | 1.1 | 2.5 |
| | | b | 3.7 | 4.2 | 3.4 | 1.7 | 1.5 | 6.1 |

The chemical recycling method of polyester fabric of the disclosure mainly uses a composite solvent to extract dye from waste PET fabric to decolorize the fabric. The decolored PET fabric contains the composite solvent. After decolorization of PET fabric containing composite solvent, ethylene glycol is directly added to depolymerize into ethylene glycol terephthalate monomer. During the entire process, the PET fabric does not need to be dried, and the depolymerization has the characteristics of low temperature and high BHET selectivity, which has low-cost benefits for the process.

The composite solvent (alcohol ether and phenyl ether) of the disclosure can simultaneously effectively decolor waste PET fabrics and accelerate the depolymerization of PET into BHET. The principle is that ethers have affinity with the PET structure. Moreover, alcohol ethers are more compatible with the EG segment monomers in the PET structure, and phenyl ethers are more compatible with the PTA segment monomers in the PET structure, making them more excellent in decolorization and depolymerization functions.

Waste PET fabrics contain impurities such as dyes and surface treatment agents. In the disclosure, a composite solvent (alcohol ether and phenyl ether) is used to extract dyes and other impurities to achieve purification effects such as decolorization. The decolored PET fabric does not need to be dried, and then depolymerized with EG into BHET monomer. Because the composite solvent plays the role of both an extraction solvent and a co-solvent for depolymerization, compared with previous technologies, PET fabrics do not need to be dried, and have high depolymerization efficiency, high BHET selectivity, and low color hue. The composite solvent is a solvent for decolorization and depolymerization at the same time. The decolorized PET fabric containing the composite solvent is depolymerized into BHET monomer in the presence of EG and catalyst to facilitate cost reduction and quality improvement. Moreover, the composite solvent used in the disclosure also has advantages in terms of environmental friendliness.

In summary, the disclosure provides a chemical recycling method for polyester fabrics, using a composite solvent (alcohol ether and phenyl ether) as the solvent for the pre-treatment of waste PET fabrics, and using the composite solvent as the depolymerization process of BHET monomer chemical recycling. The composite solvent has the functions of a solvent for pre-treatment extraction and a co-solvent for depolymerization. In this way, after using the composite solvent to decolorize the waste PET fabric, it can directly enter the depolymerization process without drying to reduce costs. The composite solvent remaining in PET fabrics is also a co-solvent for depolymerization. PET depolymerization in the presence of co-solvent has the advantages of low-temperature depolymerization, high selectivity of BHET and good color. Therefore, the quality can be improved.

What is claimed is:

1. A chemical recycling method for polyester fabrics, comprising:
performing extraction the polyester fabrics using a composite solvent and performing filtration, so as to obtain a decolored polyester fabric, the composite solvent contains alcohol ether and phenyl ether, and the decolored polyester fabric contains the composite solvent; and
adding ethylene glycol to the decolored polyester fabric for depolymerization into ethylene terephthalate monomers ethylene glycol.

2. The chemical recycling method for polyester fabrics according to claim 1, wherein alcohol ether includes ethylene glycol methyl ether, ethylene glycol ethyl ether, ethylene glycol propyl ether, ethylene glycol butyl ether, ethylene glycol phenyl ether, propylene glycol methyl ether, propylene glycol ethyl ether, propylene glycol propyl ether, propylene glycol butyl ether, propylene glycol phenyl ether or combinations thereof, Phenyl ether includes anisole, phenethyl ether, phenylpropyl ether, butyl ether, methyl anisole, methyl phenethyl ether, methyl phenyl ether, methyl phenyl ether or a combination thereof.

3. The chemical recycling method for polyester fabrics according to claim 1, wherein a weight ratio of the composite solvent to the polyester fabric is 3:10 to 10:10.

4. The chemical recycling method for polyester fabrics according to claim 1, wherein the extraction is performed at a temperature of 110° C. to 150° C.

5. The chemical recycling method for polyester fabrics according to claim 1, wherein an extraction time ranges from 10 minutes to 60 minutes.

6. The chemical recycling method for polyester fabrics according to claim 1, wherein the extraction is performed 2 times to 6 times.

7. The chemical recycling method for polyester fabrics according to claim 1, wherein in the composite solvent, a weight ratio of alcohol ether to phenyl ether is 1:9 to 9:1.

8. The chemical recycling method for polyester fabrics according to claim 1, wherein a weight ratio of ethylene glycol to the decolored polyester fabric is 2:1 to 6:1.

9. The chemical recycling method for polyester fabrics according to claim 1, wherein catalysts for depolymerization include organic metals and ionic liquids, the organic metals include zinc acetate, organic titanium, organic antimony or organic aluminum.

10. The chemical recycling method for polyester fabrics according to claim 9, wherein a weight ratio of the catalysts for depolymerization to the decolored polyester fabric is 0.5:100 to 10:100.

11. The chemical recycling method for polyester fabrics according to claim 1, wherein a temperature of depolymerization is 140° C. to 190° C.

12. The chemical recycling method for polyester fabrics according to claim 1, wherein a time of depolymerization is 1.5 hours to 5 hours.

13. The chemical recycling method for polyester fabrics according to claim 9, wherein the ionic liquids include 1-butyl-3-methylimidazolium hexa-fluoro-phosphate (BMI-PF6) and 1-butyl-3-methylimidazolium tetra-fluoro-borate (BMI-BF4).
